(19) United States
(12) Patent Application Publication
(10) Pub. No.: US 2021/0342566 A1
(43) Pub. Date: Nov. 4, 2021

(54) UNDER-DISPLAY-TYPE FINGERPRINT AUTHENTICATION SENSOR MODULE AND UNDER-DISPLAY-TYPE FINGERPRINT AUTHENTICATION DEVICE

(71) Applicants: Hiroshi ISHIBE, Kyoto City (JP); MICROMETRICS TECHNOLOGIES PTE. LTD., Primz Bizhub (SG)

(72) Inventor: Hiroshi ISHIBE, Kyoto (JP)

(21) Appl. No.: 17/271,118

(22) PCT Filed: Aug. 23, 2019
(86) PCT No.: PCT/JP2019/032963

§ 371 Date: Feb. 24, 2021

(30) Foreign Application Priority Data
Aug. 31, 2018 (JP) 2018-163157

(51) Int. Cl. G06K 9/00; H01L 27/32; H01L 51/52

(52) U.S. Cl. CPC G06K 9/0004; H01L 51/5284; H01L 27/3234

(57) ABSTRACT

An under-display-type fingerprint authenticating device includes a cover glass where a finger is placed, a display formed from an OLED provided below the cover glass, an image forming unit provided below the display and having an array of a plurality of microlenses for forming an image from the fingerprint, and a detecting module provided below the image forming unit and including an image sensor for receiving an image formed by the image forming unit. Light-shielding films are provided for preventing light from passing from the periphery of each microlens to the image sensor around the array of the plurality of microlenses, and a light-shielding dam is provided for limiting light incident from the cover glass to the array of the plurality of microlenses.

### TECHNICAL FIELD

The present invention relates to an under-display-type fingerprint authentication sensor module and an under-display-type-fingerprint authentication device, and more particularly, to an under-display-type fingerprint authentication sensor module and an under-display-type fingerprint authentication device having high resolution and small size.

### BACKGROUND ART

For a portable electronic device such as a smart phone, biometric authentication is indispensable for unlocking or other identity authentication. In particular, fingerprint authentication is dominant because of its low cost and small size. Therefore, it has been common to place a capacitive fingerprint authentication sensor in the bezel portion (periphery of the display) of a smartphone. However, with the recent trend of smartphones, the display has become larger, the bezel has disappeared, and the smartphone having a display covering the front of the smartphone has become mainstream. Therefore, it has been necessary to install the fingerprint authentication sensor at the lower part of the display.

However, the sensor does not function even if the sensor of the capacitance type or the sensor of the temperature sensing type is placed under the display, since it functions by direct contact. Types of fingerprint authentication sensors that may be functional at the bottom of the display include optical fingerprint authentication sensors and ultrasonic fingerprint authentication sensors.

On the other hand, the display is changing from a liquid crystal display to an OLED using electroluminescence, and the light passes in places other than the light emitting portion of R, G, B of the OLED, although the transmittance is about 40%. As the display passes light, the image of the finger placed on top of the display will pass through the display and the image of the fingerprint will appear on the underlying fingerprint authentication sensor. That is, in a fingerprint authentication device in which a finger, a display, and a fingerprint authentication sensor are arranged in this order (this configuration is referred to as an “under-display type”), an image of the finger is captured on the image sensor of the fingerprint authentication device. In this way, when the OLED is used as a display, a fingerprint sensor detects a fingerprint placed on the display, and the fingerprint can be authenticated.

Unlike general glasses, under the OLED light-emitting pixels, opaque light-emitting portions and wirings are provided, and generally the opaque portion is about 60% and the remaining 40% is not opaque, but the transmittance is on the order of 40%.

A portable terminal using a fingerprint authentication device having an OLED as a display is disclosed in, for example, Japanese Unexamined Patent Publication No. 2017-194676 (“Patent Document 1”). Here, a PIN diode, which operates as a fingerprint sensor, is at least partially formed in the gap between pixels in the display active area of the active matrix organic light emitting diode (AMOLED).

### CITATION LIST

### Patent Literature

Patent Document 1: Japanese Unexamined Patent Publication No. 2017-194676 (Abstract, etc.)

### SUMMARY OF THE INVENTION

### Problem to be Solved by the Invention

In a fingerprint authenticating device in a portable terminal using a conventional OLED as a display, an opaque portion exists about 60% in an OLED, and a transparent portion has only a transmittance of about 40%, and it is extremely difficult to shoot a fingerprint of a finger on the upper surface of an OLED with a fingerprint sensor module on the lower surface of the OLED. On the other hand, when the OLED is used as a display, there is a merit that a separate illuminator is not required because the OLED can be used as the illumination of the fingers.

Therefore, when the optical fingerprint authentication sensor is installed below the OLED, the OLED is not a perfect transparent body, and black opaque portions such as a light emitting portion of approximately 40 μm square are scattered, and many opaque black portions appear in front of the fingerprint image in the fingerprint image of the fingerprint authentication sensor.

On the other hand, the interval between the ridges of the fingerprint is as wide as about 250 μm, and the size of the opaque portion is as small as about 40 μm. Therefore, it is possible to leave only the ridge line of the fingerprint by passing the image of the fingerprint authentication sensor through, for example, a low-pass filter in the image processing apparatus of the subsequent stage, but in this case, there is also a problem that the resolution of the fingerprint image is lowered.

The present invention has been made in view of the above-mentioned problems, and an object of the present invention is to provide a fingerprint authentication sensor module and a fingerprint authentication device which reproduce only a fingerprint with a high resolution without the effect of a black opaque portion caused by a light emitting portion of an OLED reflected on a fingerprint or the like, even when an OLED is used as a display and a fingerprint authentication sensor is placed under the lower surface of the OLED display.

### Solutions to Problem

An under-display-type fingerprint authentication sensor module, comprising: a cover glass where a finger is placed; a display formed from an OLED which is placed below the cover glass; an image forming unit placed below the display and having an array of a plurality of microlens focusing light from the fingerprint; and a detection module placed below the image forming unit and including an image sensor to capture an image captured by the image forming unit, wherein a light-shielding film is formed that surrounds each of the plurality of microlenses and that limits light entering from the plurality of microlenses to the image sensor.

Preferably the under-display-type fingerprint authentication sensor module further comprising: an aperture means limiting incident light incident from the cover glass to the image sensor.

The aperture means may include a light-shielding dam surrounding each of the microlenses and squeezes the light from the cover glass to the array of microlenses.

The aperture means may include a plurality of apertures formed between the cover glass and the display and squeezes the light incident from the fingerprint to the image forming unit.

The image sensor may include a plurality of photoelectric conversion unit, each of the plurality of photoelectric conversion unit is disposed in a recess surrounded by a protrusion provided on the substrate, and the aperture means includes the protrusion.

The aperture means may have a depth of field at which both the fingerprint and the opaque portion of the OLED are in focus.

In another aspect of the present invention, an under-display-type finger print authentication device includes a sensor module for the under-display-type finger authentication sensor module.

### Effects of the Invention

In the fingerprint authenticating sensor module, since the light from the fingerprint is imaged by the image sensor using an array having a plurality of microlenses, even if the portion of the image of the fingerprint photographed by one microlens cannot be detected by the opaque portion of the display due to the elements included in an OLED, the portion is photographed by another microlens, and therefore, the portion is not affected by the black opaque portion due to the light emitting portion of the OLED or the like. Further, since the fingerprints are multiplexed by a plurality of microlenses, a detailed image is obtained and the resolution is increased.

As a result, even when an OLED is used as a display and the fingerprint authentication sensor is placed on the lower surface thereof, it is possible to provide a fingerprint authentication sensor module and a fingerprint authentication device that reproduce only a fingerprint with a high resolution while eliminating the effect of a black opaque portion caused by a light-emitting portion or the like of the OLED reflected on the fingerprint.

### BRIEF DESCRIPTION OF THE DRAWINGS

FIG. 1 is a cross-sectional view of a fingerprint authentication apparatus according to an embodiment of the present invention.

FIG. 2 is a plan view of a fingerprint authentication apparatus according to an embodiment of the present invention.

FIG. 3 is a diagram showing a main portion of FIG. 2.

FIG. 4 is a cross-sectional view showing a specific configuration of OLED.

FIG. 5 is a diagram showing a main part of FIG. 1.

FIG. 6 is a plan view of the portion shown in FIG. 5.

FIG. 7 is a diagram showing a specific arrangement of microlenses.

FIG. 8 is a cross-sectional view of a fingerprint authentication apparatus according to another embodiment of the present invention.

FIG. 9 is a plan view of a fingerprint authentication apparatus according to another embodiment of the present invention.

### DETAILED DESCRIPTION OF THE INVENTION

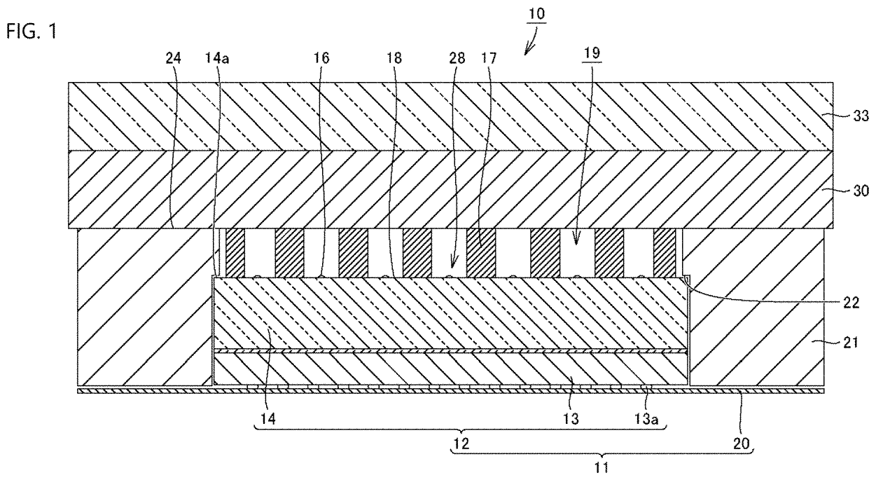

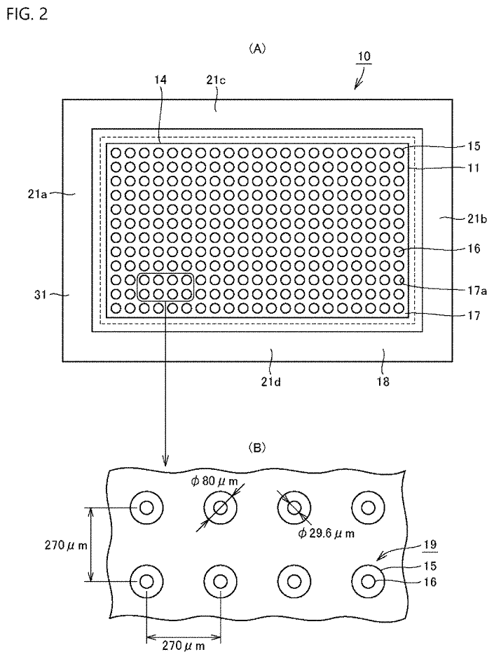

Embodiments of the present invention will be described below with reference to the drawings. FIG. 1 is a schematic cross-sectional view of an under-display-type fingerprint authentication device when an OLED according to an embodiment of the invention is used as a display, FIG. 2A is a schematic plan view of an under-display-type fingerprint authentication apparatus when the OLED shown in FIG. 1 is used as a display, and FIG. 2B is a schematic view showing a specific dimensional relation of a main part of the under-display-type fingerprint authentication apparatus.

Referring to FIGS. 1 and 2, the under-display-type fingerprint authentication device 10 when the OLED is used as a display includes a fingerprint authentication sensor module 11, an OLED 30 placed on the fingerprint authentication sensor module 11, and a cover glass 33 for placing a finger placed on an OLED 30. Fingerprint authentication sensor module 11 includes an FPC (Flexible Print Circuit) substrate 20, an image sensor 13 for capturing an image of a fingerprint connected via a land 13a thereon, a transparent glass 14 (first glass) placed on the image sensor 13, and an image forming unit 19 that is placed on the transparent glass 14 and that collects light representing fingerprints from a finger placed on the cover glass 33. The image forming unit 19 includes an array of a plurality of microlenses 16 and a thin light-shielding film 18 surrounding the periphery of the plurality of microlenses 16.

Here, a light-shielding member having a predetermined height (hereinafter, referred to as “light-shielding dam”) 17 is provided around the light-shielding film 18 surrounding the individual microlens 16. Here, the upper end portions of the light-shielding dams 17 may or may not be contacted with the OLED 30.

As shown in FIG. 2B, the microlenses 16 are arranged at a distance of 270 μm from each other in the vertical and horizontal directions, the diameter of the opening 15 formed by the light-shielding dam 17 is about 40 μm, and the diameter of the microlenses 16 is about 29.6 μm.

The image sensor 13 and the transparent glass 14 placed thereon is a rectangular parallelepiped having the same planar dimensions (referred to as the detection module 12), the fingerprint authentication sensor module 11 using the OLED as a display, and further includes a holder substrate 21 fixed for positioning the rectangular parallelepiped detection module 12 on the FPC board 20 at a predetermined position. Incidentally, FPC substrate 20 is attached to the image sensor 13 by soldering with lands 13a.

A specific method for holding the detection module 12 will be described. The four sides of an upper end of the transparent glass 14 have corners 14a. The holder substrate 21 is shaped to have a rectangular parallelepiped cavity in the center which can accommodate the image sensor 13 and the transparent glass 14. The cavity has a step 22 that divides the cavity into upper and lower parts. The opening area of the upper part of the cavity is smaller than that of the lower part of the cavity. As the step 22 presses the corners 14a of the transparent glass 14, the image sensor 13 and the transparent glass 14 are positioned by the holder substrate 21.

The holder substrate 21 has four sides, and the right, left, upper, and lower sides of the holder substrate 21 are herein denoted by 21b, 21a, 21c, 22d, respectively, for convenience. The upper end 24 of the holder substrate 21 is located higher the image forming unit 19 provided on the transparent glass 14. In the present embodiment, a space containing air is present between the transparent glass 14 and the cover glass 33 in the holder substrate 21.

The cover glass 33 on which a human finger is to be placed is disposed on the upper end 24 of the holder substrate 21.

Note that the OLED 30 does not have an illumination device for irradiating fingerprints of fingers with light because it has a light-emitting capability.

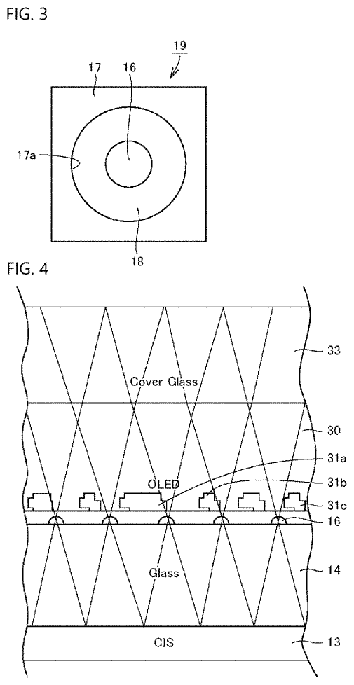

Next, the image forming unit 19 will be described. As shown in FIGS. 2A and 2B, the plurality of microlenses 16 are arranged in an array and each microlens 16 is surrounded by the light-shielding dam 17 having cylindrical inner wall surfaces 17a. The light-shielding dam 17 therefore has the plurality of circular openings 15 formed in an array as viewed from above. This will be described in detail with reference to FIG. 3.

FIG. 3 is a plan view illustrating in detail of an array around of one microlens 16 of the fingerprint authentication apparatus shown in FIG. 2A. Referring to FIG. 3, the microlens 16 is located in the center of the circular opening 15 of the light-shielding dam 17, and the region between the inner wall surface 17a of the light-shielding dam 17 and the microlens 16 is covered by the light-shielding film 18.

Next, an OLED 30 will be described. Although omitted in FIG. 1, as shown in FIG. 4 a cross-sectional view of the OLED, the OLED 30 includes a number of elements 31a, 31b, 31c, such as RGB LEDs constituting the respective pixels, and TFTs arranged at intersections of the lines arranged in the matrix direction for selecting the LEDs of the RGB. These elements 31a-31c interfere with illuminating the image sensor 13 with reflected light from a finger positioned on the cover glass 33 via a plurality of microlenses. That is, the area where the OLED 30 elements 31a-31c are present becomes opaque areas where reflected light from the fingers is not sufficiently transmitted.

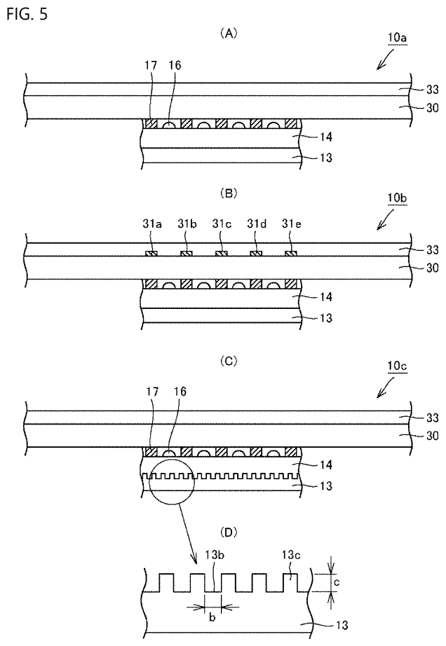

Therefore, the structure of this embodiment has a configuration wherein an image of the fingerprint is obtained with a high resolution and with a high degree of cleanliness by receiving light reflected from the fingerprint as much as possible and effectively, even in such a condition. Such a configuration will be described with reference to FIG. 5. FIG. 5A is a diagram showing a specific configuration of the first embodiment of the fingerprint authentication apparatus in this embodiment, and is basically the same as the diagram showing the central portion of FIG. 1. Here, the holder substrate 21 is not shown.

Referring to FIG. 5(A), in the first embodiment, the periphery of the individual microlenses 16 is surrounded by a light-shielding dam 17, and further the periphery of the microlens 16 is covered with a thin film so that extra light does not enter the microlens 16 and the transmission of light from the periphery of the individual microlenses to the image sensor is prevented. Therefore, only the reflected light from the fingerprint is incident on the photoelectric conversion unit of the image sensor 13.

Next, the second embodiment will be described. FIG. 5B is a diagram showing the second embodiment. Referring to FIG. 5 (B), in this embodiment, apertures 34a-34e for limiting the light incident on the microlens 16 from the fingerprint is provided between OLED 30 and the cover glass 33 thereon. The aperture is provided at a position corresponding to each of the microlens 16 arranged in a matrix.

In this embodiment, since the incident light is limited by the apertures 34 in addition to the light-shielding dam 17, it is possible to further limit unnecessary incident light.

Next, a third embodiment will be described. FIGS. 5C and 5D are diagrams showing an under-display-type fingerprint authentication device 10c according to the third embodiment. FIG. 5(C) is a cross-sectional view showing the same outline as FIG. 5(A) and FIG. 5(B), and FIG. 5(D) is an enlarged view of a portion surrounded by a circle in FIG. 5(C). Referring to FIGS. 5 (C) and (D), in this embodiment, protrusions 13c are provided surrounding the photoelectric conversion unit 13b on the image sensor 13. Thus, the light incident on the photoelectric conversion unit 13b is limited provided on the bottom surrounded by the protrusions 13c. Incidentally, the protrusions 13c are provided at positions corresponding to the individual photoelectric conversion unit 13b.

In this embodiment, since the incident light is also limited by the protrusion 13c in addition to the light-shielding dam 17, it is possible to further limit the unnecessary incident light.

Here, the height of the protrusion 13c is preferably several μm, and the ratio of the height c of the protrusion 13c to the width b of one photoelectric conversion portion 13b is preferably c/bi 1.

Next, specific methods for obtaining images that have passed through the OLED 30 will be described.

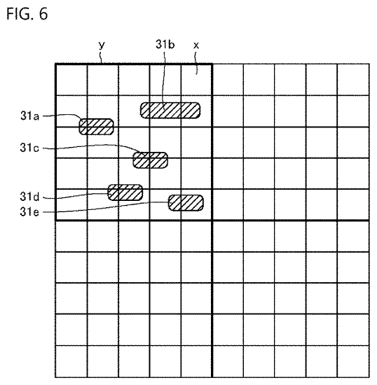

FIG. 6 is a schematic diagram of a plane illustrating an arrangement status of a plurality of elements (such as light emitting portions of LEDs and opaque TFTs) 31a to 31e provided in the OLED 30 described in FIG. 4. Here, only a part thereof is shown. Referring to FIG. 6, in this embodiment, the image sensor 13 captures a fingerprint, including the opaque portions of the OLED. In the image sensor 13, the images of the opaque portions 31a to 31c of OLED are scattered on the image of the fingerprint like dust according to the period of the light emitting portion.

Since each of the opaque portions is sufficiently smaller than the ridge spacing of the fingerprint, if the image of the fingerprint of the image sensor 13 is low-pass filtered, for example, not shown, the opaque portion of OLED disappears and only the ridge of the fingerprint remains. That is, since the size of each of the opaque portions of OLED is sufficiently higher than the frequency of the ridges of the fingerprints in terms of frequency, the opaque portions can be separated by a low-pass filter or the like.

Next, the aperture means in this embodiment will be described. In this embodiment, the above-described configuration is employed as a configuration for effectively receiving the light reflected from the fingerprint as much as possible, and thereby displaying the image of the fingerprint with a good resolution, and is referred to as “aperture means”.

That is, in this embodiment, as the aperture means, the aperture means for limiting the light incident on the image sensor 13 from the cover glass 33 is provided. The aperture means includes a light-shielding dam 17 disposed around the array of microlenses 16 and configured to squeeze light from the cover glass 33 to the array of microlenses 16, as described above.

The aperture means may also include a plurality of apertures 34a-34e disposed between the cover glass 33 and the OLED display 30 and squeezing the light from the fingerprints incident on the image forming unit 19.

Furthermore, the image sensor 13 includes a plurality of photoelectric conversion unit 13b provided on the FPC substrate 20, each of the plurality of photoelectric conversion unit 13b is disposed in a recess surrounded by a protrusion 13c provided on the FPC substrate 20 and aperture means includes the protrusions 13c.

By these aperture means, the depth of field is obtained by squeezing the aperture of the microlens. The distance between the microlens and the fingerprint and the distance between the microlens and the opaque part of the OLED are different, respectively, but by taking the depth of field of the microlens deeper, it is possible to focus both. If the subject is not in focus, the image will be out of focus and become bigger, but when the subject is in focus, the image will become sharper and smaller. Therefore, by focusing on both the fingerprint and the opaque portion of the OLED, the image becomes sharper and smaller in the opaque portion of OLED to be deleted (the frequency of the image becomes higher). Therefore, when the opaque part of the OLED is deleted by a low-pass filter or the like, the resolution of the fingerprint is kept high because the cut-off frequency of the low-pass filter can be increased. The value of the aperture for obtaining such a depth of field is preferably f=8.0 or more.

Incidentally, in this way, in order to obtain an image of a deep depth of field, not only the aperture is squeezed, the microlens 16 may be a wide-angle lens. In this case, the angle of view is preferably 60° or more.

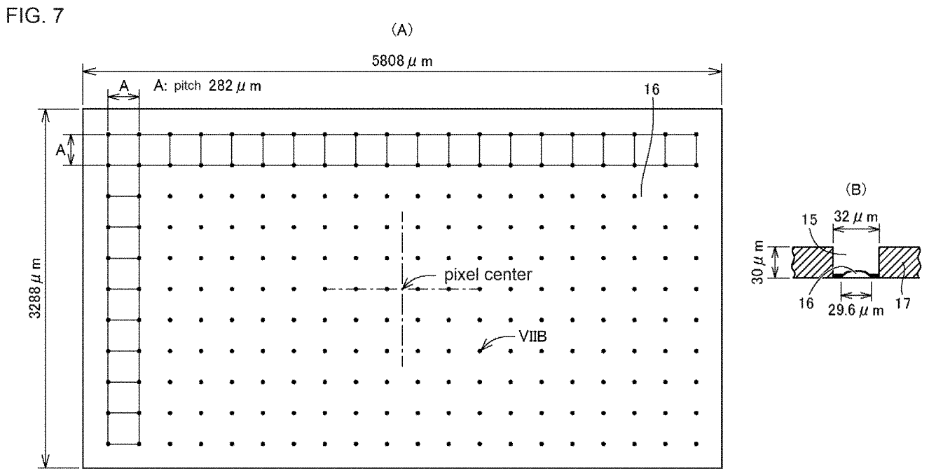

Next, another specific arrangement of the microlenses 16 that is less affected by the opaque portions of an OLED will be described. FIG. 7 is a diagram showing a specific arrangement of a microlens array comprising a plurality of microlenses 16 in this embodiment, corresponding to the previous FIG. 2. FIG. 7(A) is an entire plan view, and FIG. 7(B) is an enlarged cross-sectional view of a part where the microlens 16 shown by VIIB in FIG. 7(A) is located.

Referring to FIG. 7A, in this embodiment, holes having a pitch of 282 μm are provided in both the horizontal and vertical directions wherein the number of holes in the horizontal direction is 20, whereas that of the vertical directions is 11 on a surface having a width of 5808 μm and a length of 3288 μm on the first glass 14, and the microlenses 16 are arranged therein. In the figure, the pixel center is indicated by a cross.

Thus, since the light from the fingerprint is imaged by the image sensor using an array having a plurality of microlenses, even if the portion captured by one of the microlens of the images of the fingerprint cannot be detected, the portion is captured by the other microlens, the image is not affected by the black opaque portion such as the light emitting portion of the OLED. Further, since the fingerprints are multiplexed by a plurality of microlenses, a detailed image is obtained and the resolution is increased.

Note that the pixel center is the center of the pixels (center) of the image sensor 13. When placing the microlenses 16, they are arranged in the vertical and horizontal directions from the pixel center of the image sensor 13. That is, the microlenses 16 are arranged symmetrically, with respect to the pixel center in horizontal and vertical directions.

Further, with reference to FIG. 7 (B), the diameter of the opening 15 where a microlens 16 is provided is 32 μm and small as compared with FIG. 2, the depth is 30 μm, and the ratio of the diameter and depth is approximately 1 to 1. The diameter of the microlens is 29.6 μm.

Next, another embodiment of the present invention will be described.

In another embodiment, a fingerprint authenticator does not have an OLED 30 and uses a transparent glass 35 instead of an OLED 30. In this example, LEDs 36 for illuminating fingerprints is separately provided. This may be arranged, for example, by providing concave portions on the holder substrate 21 or the like.

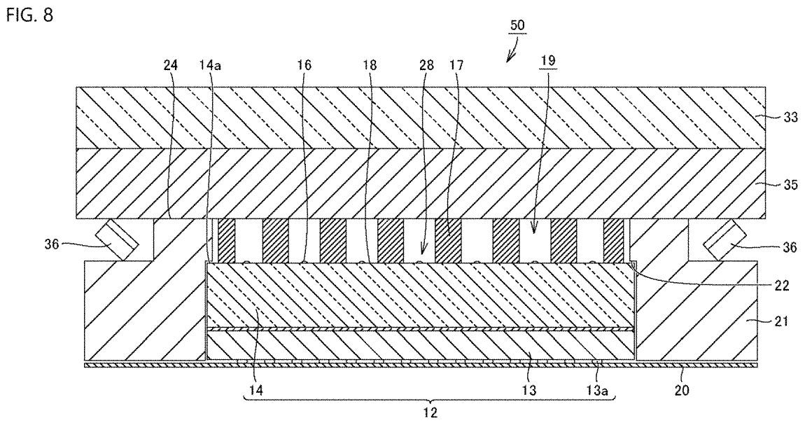

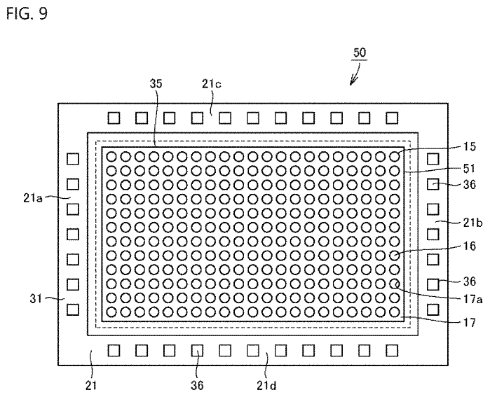

FIGS. 8 and 9 show the configuration of this embodiment. FIG. 8 is a cross-sectional view corresponding to FIG. 1 in this embodiment, and FIG. 9 is a plan view corresponding to FIG. 2. Referring to FIGS. 8 and 9, in this embodiment, a transparent glass 35 is provided instead of the OLED 30 of FIGS. 1 and 2. LEDs 36 for illuminating the fingerprints are also provided separately. The other parts are the same as those in FIGS. 1 and 2, and therefore description thereof is omitted. In this embodiment, the fingerprint authentication device is denoted by 50, and the fingerprint authentication sensor module is denoted by 51.

In this embodiment, since there is no light-shielding member inside instead of the OLED 30, in addition to obtaining enough reflected light from the fingerprint, it is possible to obtain an image of a sharper fingerprint because unnecessary reflected light can be eliminated.

In the above embodiments, to obtain as much reflected light from the fingerprints as possible, the configuration for effectively receiving light has been described individually for the first to third three embodiments, these embodiments may be arbitrarily combined.

In the above embodiment, the configuration in which an OLED is used as a display and the opaque portion of the OLED has been described, but the present invention can be similarly applied to a display having the opaque portion as in the OLED.

Although the embodiments of the present invention are described above with reference to the accompanying drawings, the present invention is not limited to the illustrated embodiments. Various changes and modifications can be made to the illustrated embodiments without departing from sprit and the scope of the present invention.

### INDUSTRIAL APPLICABILITY

The fingerprint authentication device according to the invention embodiment is advantageously used as a fingerprint authentication device having an OLED because a fingerprint authentication device capable of effectively performing fingerprint authentication can be obtained even when a fingerprint authentication sensor is placed on the lower surface of an OLED.

### REFERENCE SIGNS LIST

10, 30 OLED fingerprint authentication device, 11, 51 fingerprint authentication sensor module, 12 detecting module, 13 image sensor, 13a land, 13b photoelectric converter, 13c projection, 14 transparent glass, 14a corner, 15 opening, 16 microlens, 17 light-shielding dam, 18 light-shielding film, 19 image forming unit, 20 FPC substrate, 21 holder substrate, 22 step portion, 30 OLED, 31 device, 33 cover glass, 34 aperture, 35 transparent glass, 41 light-shielding member, 50 fingerprint authentication device.

1. An under-display-type fingerprint authentication sensor module, comprising:
a cover glass where a finger is placed;
a display formed from an OLED which is placed below the cover glass;
an image forming unit placed below the display and having an array of a plurality of microlens focusing light from the fingerprint; and
a detection module placed below the image forming unit and including an image sensor to capture an image captured by the image forming unit, wherein
a light-shielding film is formed that surrounds each of the plurality of microlenses and that limits light entering from the plurality of microlenses to the image sensor.

2. The under-display-type fingerprint authentication sensor module according to claim 1, further comprising:
an aperture means limiting incident light incident from the cover glass to the image sensor.

3. The under-display-type fingerprint authentication sensor module according to claim 2, wherein
the aperture means includes a light-shielding dam surrounding each of the microlenses and squeezes the light from the cover glass to the array of microlenses.

4. The under-display-type fingerprint authentication sensor module according to claim 2, wherein
the aperture means includes a plurality of apertures formed between the cover glass and the display and squeezes the light incident from the fingerprint to the image forming unit.

5. The under-display-type fingerprint authentication sensor module according to claim 2, wherein
the image sensor includes a plurality of photoelectric conversion unit,
each of the plurality of photoelectric conversion unit is disposed in a recess surrounded by a protrusion provided on the substrate, and
the aperture means includes the protrusion.

6. The under-display-type fingerprint authentication sensor module according to claim 2, wherein the aperture means has a depth of field at which both the fingerprint and the opaque portion of the OLED are in focus.

7. An under-display-type finger print authentication device including a sensor module for the under-display-type finger authentication sensor module according to claim 1.
